(19) United States
(12) Patent Application Publication
(10) Pub. No.: US 2009/0257998 A1
(43) Pub. Date: Oct. 15, 2009

(54) METHOD FOR TREATING HEART ATTACKS, STROKES AND DIABETIC CRISIS BY UTILIZING MODIFIED VIRUS VIRIONS TO INSERT NECESSARY PROTEIN MOLECULES AND VITAL NUTRIENTS INTO TARGETED CELLS

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 12/101,988

(22) Filed: Apr. 12, 2008

(51) Int. Cl. A61K 38/43; A61K 38/16; A61K 38/45; A61K 38/52; A61K 38/51; A61K 38/44; A61K 38/53; A61K 33/00; A61K 31/70; A61K 31/20; A61K 31/7052; A61P 9/00; A61P 3/10

(52) U.S. Cl. 424/94.4; 514/12; 424/94.1; 424/94.5; 424/613; 514/23; 514/558; 514/44 R

(57) ABSTRACT

The common link between incurring a heart attack, suffering a stroke or diabetic crisis is related to deficiencies involving a body's metabolism of glucose and the ability to optimally conduct the necessary biologic processes of aerobic respiration to produce energy molecules. Utilizing a medical treatment method comprised of a modified form of virus to deliver to cells in the body the protein molecules and nutrients needed to enhance the processes of glycolysis, the tricarboxylic acid cycle, oxidative phosphorylation and anaerobic respiration will save lives. Providing an alternative means for brain cells and heart muscle cells to have access to proteins, nutrients such as glucose and oxygen, and energy molecules by providing these tissues with viruses that carry these vital elements to these tissues will greatly improve the survivability of individuals experiencing a heart attack, a stroke or a diabetic crisis.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2008 Lane B. Scheiber and Lane B. Scheiber II. A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

Any medical treatment method intended to correct a protein or nutrient deficiency in the body by utilizing a modified virus to insert a quantity of protein molecules or a quantity of nutrient molecules into one or more cells of the body.

2. Description of Background Art

The medical conditions where a heart attack or a stroke occurs generally is associated with or the result of a lack of sufficient supply of oxygenated blood to the heart in the case of a heart attack or to the brain in case of a stroke. One of the vital functions of blood is to act as the vehicle, by way of hemoglobin residing in the red blood cells that circulate in blood, to carry oxygen to the tissues where individual cells can utilize the oxygen for the purpose of aerobic respiration. Other important functions of blood include supplying the body's cells with nutrients such as glucose and the removal of waste products such as carbon dioxide from cells. Oxygen becomes critical as demonstrated in the brain, where at a normal body temperature, brain cells cannot be without a sufficient supply of oxygen for more than five minutes before irreversible damage to brain cells begins to occur. Other tissues in the body, including heart cells, demonstrate a chronic need for an adequate supply of oxygen, and when not enough oxygen is supplied to the tissues of the body, damage occurs to those cells. If an ample amount of oxygen is unable to reach and nourish cells in the body in a timely fashion, cells die for lack of being able to generate a sufficient amount of energy to sustain life.

Oxygen and glucose are two vital consumable nutrients that provide all cells in the body the raw materials to generate energy in the form of adenosine triphosphate (ATP). ATP is the common energy medium used throughout the cell, and in all cells of the body, to power the biologic processes that sustain life in the cell. Both oxygen and glucose are transported by the blood to the cells of the body. Inside the cell, glucose is transformed by the biologic process of glycolysis into pyruvate. For generation of maximum number of ATP molecules from the parent molecule glucose, pyruvate then is metabolized by the tricarboxylic acid cycle and oxidative phosporylation. Oxygen is vital participant in oxidative phosphorylation. When sufficient oxygen is available to the cell, by means of aerobic respiration one glucose molecule can yield 36 ATP molecules in nerve and muscle cells, as many as 38 ATP molecules in liver and heart cells. In circumstances where an insufficient amount of oxygen is available to the cell, pyruvate is diverted to an anaerobic respiration process and is converted to lactic acid by the enzyme lactate dehydrogenase. The conversion of pyruvate to lactic acid yields 2 ATP molecules.

Diabetic crisis occurs due to cells not being able to sufficiently utilize circulating glucose. The level of glucose rises in the blood due to the cells not being capable of adequately transporting glucose efficiently from the blood into cells, resulting in the cells of the body not being able to efficiently process the glucose through aerobic respiration including glycolysis, tricarboxylic acid cycle and oxidative phosporylation to produce energy molecules.

A method of treatment to reduce damage to cells at times when there exits a state where a lack of sufficient aerobic respiration threatens the health of cells due to an adequate supply of oxygenated blood, such as a heart attack, stroke or diabetic crisis, is to optimize the overall respiration process or increase the anaerobic respiration process or provide an alternate means to supply the vital nutrients such as oxygen and glucose or energy molecules to the cells being threatened. The respiration process is comprised of a chain of biologic reactions that occur due to the presence of enzymes that catalyze the reactions. Increasing the number of available enzymes that would participate in the respiration process would help to maximize the utilization of glucose in cells that are stressed.

For aerobic respiration, which is dependent upon the presence of both oxygen and glucose, increasing only the number of enzymes would force a utilization of all available oxygen and glucose inside the cell, but aerobic respiration would cease once the consumable nutrients were utilized. Two options to optimize survival of cells threatened by a lack of sufficient oxygen supplied by blood are to increase the efficiency of a means of producing energy that is not dependent upon oxygen, therefore increase the output of the anaerobic respiratory process inside the threatened cells, or provide an alternative means of supplying the cells with oxygen and glucose or energy molecules. Supplying cells cut off from a supply of oxygenated blood, with an alternative supply of energy molecules, could sustain such threatened cells until the deficiency in the supply of oxygenated blood is corrected.

A method of optimizing the respiratory process inside cells would be to utilize modified virus virions as a vehicle to transport necessary enzymes, nutrients such as oxygen and glucose molecules and even energy molecules to cells threatened by a lack of oxygen during periods of crisis such in the event of a heart attack, stroke or diabetic crisis.

A virus is an obligate parasite. A virus is comprised of generally one or two shells, glycoprotein probes affixed to the outermost shell, one or more genetic material and in some cases enzymes to assist in the replication process are carried in the core of the virus. An intact, individual form of a virus, as it exists outside the boundaries of a host cell, is generally referred to as a ‘virion’.

Glucose, a six-carbon molecule, represents a form of sugar. Glucose is absorbed by the cells of the body through passive diffusion and is converted to energy by the biologic processes of glycolysis, the tricarboxylic acid (TCA) cycle and oxidative phosporylation. Insulin, a protein, facilitates the absorption of glucose into cells. Normal range for blood glucose in humans is generally defined as a fasting blood plasma glucose level of between 70 to 110 mg/dl.

Glucose generally enters the body and then the blood stream as a result of the digestion of food. For purposes of this description, ‘blood’ and ‘blood stream’ refer to the same substance, which is generally considered to be blood as a whole including plasma and blood cells. The beta cells of the Islets of Langerhans continuously sense the level of glucose in the blood and respond to elevated levels of blood glucose by secreting insulin into the blood. Beta cells produce the protein ‘insulin’ in the endoplasmic reticulum and store the insulin in vacuoles until the insulin is needed. When beta cells detect an increase in the glucose level in the blood, the beta cells release insulin into the blood plasma from said storage vacuoles.

Insulin is a protein; proteins are comprised of amino acids. An insulin molecule consists of two chains of amino acids, an alpha chain and a beta chain, linked by two disulfide (S—S) bridges. The alpha chain consists of 21 amino acids. The beta chain consists of 30 amino acids.

A eukaryote refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure cell-surface receptors that the cell uses to detect properties of the environment surrounding the cell membrane. Cytoplasm refers to the entire contents inside the cell except for the nucleus and acts as a filling medium inside the boundaries of the plasma cell membrane. Cytosol refers to the semifluid portion of the cytoplasm minus the mitochondria and the endoplasmic reticulum. The nucleus, organelles, and ribosomes are suspended in the cytosol. Nutrients such as amino acids, oxygen and glucose are present in the cytosol. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex. Floating in the cytoplasm, but also located in the endoplasmic reticulum and mitochondria are ribosomes. Ribosomes are protein structures comprised of several strands of proteins that combine and couple to a messenger ribonucleic acid (mRNA) molecule. More than one ribosome may be attached to a single mRNA at a time. Ribosomes decode genetic information coded in a mRNA molecule and manufacture proteins to the specifications of the instruction code physically present in the mRNA molecule.

The majority of the deoxyribonucleic acid (DNA) in a cell is present in the form of chromosomes, the double stranded helical structures located in the nucleus of the cell. DNA in a circular form, can also be found in the mitochondria, the powerhouse of the cell, an organelle that assists in converting glucose into usable energy molecules. DNA represents the genetic information a cell needs to manufacture the materials it requires to develop to its mature form, sustain life and to replicate. Genetic information is stored in the DNA by arrangements of four nucleotides referred to as: adenine, thymine, guanine and cytosine. DNA represents instruction coding, that in the process known as transcription, the DNA's genetic information is decoded by transcription protein complexes referred to as polymerases, to produce ribonucleic acid (RNA). RNA is a single strand of genetic information comprised of coded arrangements of four nucleotides: adenine, uracil, guanine and cytosine. The physical difference in the construction of a DNA molecule versus an RNA molecule is that DNA utilizes the nucleotide ‘thymine’, while RNA molecules utilize the nucleotide ‘uracil’. RNAs are generally classified as messenger RNAs (mRNA), transport RNAs (tRNA) and ribosomal RNAs (rRNA).

Mitochondrion (‘mitochondria’ pleural form) is a cellular organelle that is considered the energy producing organelle of the cell. Mitochondria assist in generating energy for cell metabolism by producing ATP molecules from glucose. Within the cytoplasm and outer wall of the mitochondria sugar molecules undergo the process of glycolysis, and then inside the mitochondria byproducts of glucose are further broken down in the tricarboxylic acid (TCA) cycle and by oxidative phosporylation to useable energy molecules.

The exterior of a mitochondrion is known as an external membrane. Inside the outer membrane is an inner membrane. Folds in the inner membrane create crista, which expands the surface of the inner membrane and enhances the mitochondrion's ability to create the energy molecules referred to as adenosine triphosphate molecules or ATP molecules. Inside the inner membrane is the mitochondrion matrix. The mitochondrion matrix contains a highly concentrated mixture of enzymes, ribosomes, tRNA and mitochondrial DNA. Glycolysis occurs in the cytosol of the cell and membrane of the mitochondrion. The tricarboxylic acid cycle functions within the inner chambers and matrix of the mitochondrion. Oxidative phosporylation occurs within the outer and inner membranes of the mitochondrion.

Many of the intermediates of the processes of glycolysis and the tricarboxylic acid cycle exist as anions at the pH found in cells, and readily associate with H⁺ to form acids. The intermediates of glycolysis and the tricarboxylic acid cycle are therefore often written as either an anion or an acid. For the purposes of this description, the intermediates of the processes of glycolysis and the tricarboxylic acid cycle are generally written as anions (as an example pyruvate versus pyruvic acid).

As a result of the biochemical process of glycolysis during aerobic (oxygen sufficiently available) respiration conditions glucose is converted to pyruvate. The abbreviated processes of glycolysis include: (1) Glucose is converted to glucose-6-phosphate by the enzyme ‘hexokinase’. (2) Glucose-6-phosphate is converted to fructose-6-phosphate by the enzyme ‘glucose-6-phosphate isomerase’. (3) Fructose-6-phosphate is converted to fructose 1,6-diphosphate by the enzyme ‘phosphofructo kinase’. (4) Fructose 1,6-diphosphate is converted to two different entities including dihydroxyacetone-3-phosphate and glyceraldehydes-3-phosphate by the enzyme ‘fructose bisphosphate aldolase’. (5) Dihydroacetone-3-phosphate converts to D-glyceraladehyde-3-phosphate by the enzyme ‘triose-phosphate isomerase’. (6) Glyceraldehyde-3-phosphate is converted to 1,3-diphosphoglycerate by the enzyme ‘glyceraldehyde-3-phosphate dehydrogenase’. (7) 1,3-diphosphoglycerate is converted to 3-phosphoglycerate by the enzyme ‘phosphoglycerate kinase’. (8) 3-phosphoglycerate is converted to 2-phosphoglycerate by the enzyme ‘phosphoglycerate mutase’. (9) 2-phosphoglycerate is converted to phosphoenolpyruvate by the enzyme ‘enolase’. (10) Phosphoenolpyruvate is converted to pyruvate by the enzyme complex referred to as ‘pyruvate kinase’.

Pyruvate is then oxidized to an acetyl group, which is combined with Coenzyme A and produces acetyl Coenzyme A (acetyl-CoA). Pyruvate dehydrogenase, which metabolizes pyruvate to acetyl-CoA is comprised of a multi-enzyme complex. The three protein complexes of pyruvate dehydrogenase are designated E1 (pyruvate dehydrogenase), E2 (dihydrolipoamide S-acetyltransferase), and E3 (dihydrolipoamide dehydrogenase). Acetyl-CoA enters the tricarboxylic acid cycle. Under aerobic respiration conditions from one glucose molecule the process of glycolysis generates 8 ATP molecules, conversion of pyruvate to acetyl CoA generates an additional 6 ATP molecules.

The tricarboxylic acid cycle otherwise known as the citric acid cycle or the Krebs cycle, was discovered in 1937 by Sir Hans Krebs, and is a biochemical process that provides complete oxidation of acetyl-CoA, which may be derived from sources such as fats, carbohydrates and lipids. For purposes of this discussion, acetyl-CoA is a byproduct of glucose metabolism during glycolysis, and enters the tricarboxylic acid cycle and (1) combines with oxaloacetate (also referred to as oxaloacetic acid) by the action of the enzyme ‘citrate synthetase’ which produces citrate (also referred to as citric acid). (2) Citrate is converted to cis-aconitate (also referred to as cis-aconitic acid) per the enzyme ‘aconitase’. (3) Cis-aconitate is converted to iso-citrate (also referred to as isocitric acid) again by the enzyme aconitase. (4) Iso-citrate is converted to alpha-ketoglutarate (also referred to as alpha-ketoglutaric acid) by the enzyme ‘isocitrate dehydrogenase’. (5) Alpha-ketoglutarate is converted to succinyl CoA by the enzyme ‘2-oxoglutarate dehydrogenase’. (6) Succinyl CoA is converted to succinate (also referred to as succinic acid) by the enzyme ‘succinyl-CoA synthetase’. (7) Succinate is converted to fumarate (also referred to as fumaric acid) by the enzyme ‘succinate dehydrogenase’. (8) Fumarate is converted to malate (also referred to as malic acid) by the enzyme ‘fumarate hydratase’. (9) Malate is converted to oxaloacetate by the enzyme ‘malate dehydrogenase’. The result of metabolism of glucose by glycolysis and the tricarboxylic acid cycle yields ATP molecules and electron donor molecules such as the reduced form of the coenzyme nicotinamide adenine dinucleotide written NADH +H⁺. The tricarboxylic acid cycle also produces electron donor molecules in the form of the reduced co-enzyme flavin adenine dinucleotide written FADH₂.

Oxidative phosphorylation is a metabolic pathway that uses energy released by oxidation to produce adenosine triphosphate (ATP) molecules. During oxidative phosporylation electrons are transferred from electron donors to electron acceptors such as oxygen in redox reactions. In eukaryotes the redox reactions are carried out by a series of protein complexes located within mitochondria. These protein complexes represent a linked set of enzymes referred to as electron transport chains. The protein complexes utilized in oxidative phosphorylation include nicotinamide adenine dinucleotide (NADH) dehydrogenase enzyme molecule, the succinate dehydrogenase enzyme molecule, the cytochrome-c reductase enzyme molecule, the cytochrome-c oxidase enzyme molecule, and the ATP synthase enzyme molecule. Under aerobic respiration conditions one glucose molecule metabolized by the combination of glycolysis, the tricarboxylic acid cycle and oxidative phosphorylation yields as many as 38 ATP molecules in liver and heart cells; 36 ATP molecules in some tissues such as nerve and muscle cells.

An enzyme is a protein generated by cells that acts as a catalyst to induce chemical changes in other substances, itself remaining apparently unchanged in the process. There are several main groups of enzymes including oxidoreductase, transferase, hydrolase, lyase, isomerase, and ligase, sometimes referred to as synthetase. EC is an abbreviation for Enzyme Commission of the International Union of Biochemistry and this is used in conjunction with a unique number to define a specific enzyme identified in the Enzyme Commission's list of enzymes. Oxidoreductases generally have as their first EC identifying number, the number 1. Transferases generally have as their first EC identifying number, the number 2. Hydrolases generally have as their first EC identifying number, the number 3. Lyases generally have as their first EC identifying number, the number 4. Isomerase generally have as their first EC identifying number, the number 5. Ligases generally have as their first EC identifying number, the number 6. Several scientific names often exist to identify the same enzyme.

Enzymes (followed by their Enzyme Commission of the International Union of Biochemistry number) utilized in the metabolism of glucose in the processes of glycolysis, tricarboxylic acid cycle, and oxidative phosporylation are described in the following paragraphs.

Hexokinase (EC 2.7.1.1), is also referred to as hexokinase type IV glucokinase or in some cases simply glucokinase. Hexokinase converts glucose to glucose-6-phosphate in glycolysis.

Glucose-6-phosphate isomerase (EC 5.3.1.9), is also known as phosphoglucoisomerase. Glucose-6-phosphate isomerase is an enzyme that converts glucose-6-phosphate to fructose-6-phosphate in glycolysis.

6-phosphofructokinase (EC 2.7.1.11), is also known as phosphofructokinase. 6-phosphofructokinase is an enzyme that converts fructose-6-phosphate to fructose 1,6-diphosphate in glycolysis.

Fructose bisphosphate aldolase (EC 4.1.2.13), is also known as aldolase. Fructose bisphosphate aldolase is an enzyme that converts fructose 1,6-diphosphate to two different entities including dihydroxyacetone 3-phosphate and glyceraldehydes 3-phosphate in glycolysis.

Triose-phosphate isomerase (EC 5.3.1.1). Triose-phosphate isomerase is an enzyme that converts dihydroacetone-3-phosphate converts to D-glyceraladehyde-3-phosphate.

Glyceraldehyde-3-phosphate dehydrogenase (EC 1.2.1.12), may be abbreviated GAPDH or G3PDH. Glyceraldehyde-3-phosphate dehydrogenase is an enzyme that converts glyceraldehydes-3-phosphatate to 1,3-diphosphoglycerate in glycolysis.

Phosphoglycerate kinase (EC 2.7.2.3). Phosphoglycerate kinase is an enzyme that converts 1,3-diphosphoglycerate to 3-phosphoglycerate in glycolysis.

Phosphoglycerate mutase (EC 5.4.2.1). Phosphoglycerate mutase is an enzyme that converts 3-phosphoglycerate to 2-phosphoglycerate in glycolysis.

Enolase (EC 4.2.1.11). Enolase is an enzyme that converts 2-phosphoglycerate to phosphoenolpyruvate in glycolysis.

Pyruvate kinase (EC 2.7.1.40). Pyruvate kinase is an enzyme that converts phosphoenolpyruvate to pyruvate in glycolysis.

Pyruvate dehydrogenase. Pyruvate dehydrogenase is comprised of three units. The three units include E1 (EC 1.2.4.1), (EC 1.2.1.51), E2 dihydrolipoamide S-acetyltransferase (EC 2.3.1.12), and E3 dihydrolipoamide dehydrogenase (EC 1.8.1.4). Pyruvate dehydrogenase molecular complex catalyzes the conversion of pyruvate to acetyl-CoA.

Citrate synthetase (EC 4.1.3.7). Citrate synthetase is an enzyme that converts acetyl-CoA combines with oxaloacetate to produce citrate in the tricarboxylic acid cycle.

Aconitase (EC 4.2.1.3). Aconitase exists in two isoenzyme forms in eukaryotes: mitochondrial and cytosolic. Aconitase is an enzyme that converts citrate to cis-aconitate in the tricarboxylic acid cycle and converts cis-aconitate to iso-citrate in the tricarboxylic acid cycle.

Isocitrate dehydrogenase (EC 1.1.1.41). Isocitrate dehydrogenase is an enzyme that converts isocitrate to alpha-ketoglutaric acid in the tricarboxylic acid cycle.

2-oxoglutarate dehydrogenase. 2-oxoglutarate dehydrogenase is a protein complex comprised of three units. The three units include E1 (EC 1.2.4.2), E2 (EC 2.3.1.61), and E3 (EC 1.8.1.4). 2-oxoglutarate dehydrogenase is an enzyme complex that converts alpha-ketoglutaric to succinyl CoA in the tricarboxylic acid cycle.

Succinyl-CoA synthetase (EC 6.2.1.5). Succinyl-CoA synthetase is an enzyme that converts succinyl CoA to succinate in the tricarboxylic acid cycle.

Succinate dehydrogenase (EC 1.3.5.1). Succinate dehydrogenase is an enzyme that converts succinate to fumarate in the tricarboxylic acid cycle.

Fumarate hydratase (EC 4.2.1.2). Fumarate hydratase is an enzyme that converts fumarate to malate in the tricarboxylic acid cycle.

Malate dehydrogenase (EC 1.1.1.37). Malate dehydrogenase is an enzyme that converts malate to oxaloacetate in the tricarboxylic acid cycle.

During conditions were sufficient oxygen is available, metabolism of glucose generally occurs through the processes of glycolysis, tricarboxylic acid cycle, and oxidative phosporylation. When oxygen is not readably available, anaerobic respiration occurs. The enzyme lactate dehydrogenase provides an alternative pathway to produce ATP when sufficient oxygen is not available by converting pyruvate to lactic acid. The anaerobic pathway lactate dehydrogenase catalyzes is much less efficient means of producing energy molecules than the aerobic pathway that takes advantage of oxygen dependent processes of tricarboxylic acid cycle and oxidative phosporylation.

Proteins are comprised of a series of amino acids bonded together in a linear strand, sometimes referred to as a chain; a protein may be further modified to be a structure comprised of one or more similar or differing strands of amino acids bonded together. A protein comprised of more than one strand of amino acids (referred to as subunits) may be referred to as a protein complex. Insulin is a protein structure comprised of two strands of amino acids, one strand comprised of 21 amino acids long and the second strand comprised of 30 amino acids; the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors.

Messenger RNAs (mRNA) are created by transcription of DNA. Messenger RNA generated by transcription of nuclear DNA, migrate out of the nucleus of the cell, and are utilized as protein manufacturing templates by ribosomes. Different mRNAs code for different proteins. As previously mentioned, there are as many as 30,000 varieties of proteins, therefore there are at least 30,000 different mRNA molecules. A ribosome is a protein complex that manufactures proteins by deciphering the instruction code located in a mRNA molecule. When a specific protein is needed, pieces of the ribosome complex bind around the strand of mRNA that carries the specific instruction code that will generate the required protein. The ribosome traverses the mRNA strand and deciphers the genetic information coded into the sequence of nucleotides that comprise the mRNA molecule and generates the protein by binding together amino acids into a chain.

Viruses are obligate parasites. Viruses simply represent a carrier of genetic material and by themselves viruses are unable to replicate or carry on any form of biologic function outside their host cell. Viruses are generally comprised of one or more shells constructed of one or more layers of protein or lipid material, and inside the outer shell or shells, carries a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell is a cell that provides the virus the proper biochemical machinery for the virus to successfully replicate itself.

Protected by the outer coat generally comprised of an envelope or capsid or envelope and capsid, viruses carry a genetic payload in the form of deoxyribonucleic acid (DNA) or ribonucleic acid (RNA). Once a virus's exterior probes locate and functionally engage the surface receptor or receptors on a host cell, the virus inserts its genetic payload into the interior of the host cell. In the event a virus is carrying a DNA payload, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate their RNA being converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the host cell's nuclear DNA and simply utilize portions of its viral genome to act as messenger RNA (mRNA). RNA viruses that bypass the host cell's DNA, cause the cell, in general, to generate copies of the necessary parts of the virus directly from the virus's RNA genome.

The Hepatitis C virus (HCV) is a positive sense RNA virus, meaning a type of RNA that is capable of bypassing the need for involving the host cell's nucleus by having its RNA genome function as messenger RNA. Hepatitis C virus infects liver cells. The Hepatitis C viral genome becomes divided once it gains access to the interior of a liver host cell. Portions of the subdivisions of the Hepatitis C viral genome directly interact with host liver cell's ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to activate the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

As an alternative example of how virus's function to locate their host, the Human Immunodeficiency Virus seeks out and engages its host, the T-Helper cell, by utilizing its glycoprotein 120 probe and glycoprotein 41 probe to intercept the CD4 cell-surface receptor and either the CCR5 or CXCR4 cell-surface receptor on the T-Helper cell.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construction copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus offers a naturally occurring vehicle mechanism to transport and insert medically therapeutic protein molecules, nutrient molecules or energy molecules into liver cells and other specifically targeted cells of the human body. The naturally occurring Hepatitis C virus already is equipped with the means of seeking out liver cells and delivering to liver cells its genetic payload. Further, the surface probes present on the Hepatitis C virus's outer protein coat can be modified to seek out specific receptors on specific target cells other than liver cells. Once the modified Hepatitis C virus's probes properly engage the cell-surface receptors on a target cell, the modified Hepatitis C virus would insert into the target cell its payload of medically therapeutic protein molecules, nutrient molecules or energy molecules for the purpose of achieving a medically therapeutic response.

The Hepatitis C virus is one of several viruses that have been identified that possess the natural capacity to locate and infect liver cells with the genome the virus carries, thus including a liver cell as part of its reproductive cycle. Hepatitis A virus (HAV), Hepatitis C virus (HCV), Hepatitis D virus (HDV), Hepatitis E virus (HEV), and Hepatitis G virus (HGV) have been identified to carry their genome as RNA. The Hepatitis G virus is considered to be very similar to the Hepatitis C virus. The Hepatitis F virus and Hepatitis H viruses at this point are not considered to exist, though this is controversial. The Hepatitis B virus (HBV) is believed to carry its genome as DNA. These alternative hepatitis viruses may also be utilized to act as alternative vehicles to deliver medically therapeutic protein molecules, nutrients or energy molecules to liver cells or specific target cells.

Current state of gene therapy generally refers to efforts directed toward inserting an exogenous subunit of DNA into a vehicle such as a virus. The vehicle is intended to insert the exogenous subunit of DNA into a target cell. The exogenous DNA subunit then migrates to the target cell's nucleus. The exogenous DNA subunit then inserts into the native DNA of the cell. This represents a permanent alteration of the cell's nuclear DNA. The nuclear transcription proteins then read the exogenous DNA subunit's nucleotide coding to generate RNA molecules to produce the intended cellular response. The approach described within the scope of this text involves protein molecules, nutrients or energy molecules versus DNA as a modified virus virion's payload. DNA is comprised of the nucleotides adenine, thymine, guanine and cytosine. RNA is composed of the nucleotides adenine, uracil, guanine and cytosine. DNA codes for the manufacture of RNAs, which are composed of nucleotides. Messenger RNAs code for the manufacture of proteins, which are composed of amino acids. Transport RNAs transport single amino acids to ribosomes. Ribosome RNAs assist with the construction of ribosomes. Proteins are comprised of amino acids. Oxygen is an element. Glucose is a sugar molecule. Energy molecules such as adenosine triphosphate are nucleotide that acts as an energy source. Protein molecules, oxygen, glucose and adenosine triphosphate are physically and functionally different than DNA.

If the mitochondria's energy producing mechanisms fail to operate at an optimal level, overall cell function suffers due to a decline in the supply of available energy. Glucose may indeed be available for utilization by the mitochondria, but actual utilization rate of the glucose will be reduced if the cell's mitochondria are not functioning properly, with the result being the necessary supply of ATP molecules may not be available for the cell to function properly as required.

A Hepatitis C virus modified to carry therapeutic protein molecules could be introduced into the blood stream, travel the blood stream, engage the receptors on a liver cell with its surface probes, and then insert the protein molecules it carries as a payload into the liver cells. A payload consisting of a quantity of protein molecules could be used to enhance cellular biochemical reactions in liver cells deficient in a particular protein. Hepatitis C viruses could be further modified to not only carry protein molecules instead of its own RNA genome, but also modified such that the surface probes of the Hepatitis C virions are altered to seek out and engage the surface-receptors on specific cells in the body. Modified Hepatitis C virus virions could be fashioned to deliver medically therapeutic protein molecules, nutrient molecules and energy molecules to specific cells in the body.

Minutes may be vital to the survival of an individual suffering from a heart attack or a stroke or a diabetic crisis. When cells experience a lack of oxygenated blood such cells run the risk of incurring irreversible damage. Up until now there has been no alternative means to oxygenate cells that are becoming compromised due to a lack of supply of sufficient oxygenated blood to provide needed oxygen and glucose to cells at risk of sustaining irreversible damage to facilitate aerobic respiration in such cells. Utilizing modified virus virions to transport protein molecules, nutrients such as oxygen and glucose, or energy molecules such as ATP, offers an alternate means to relying on oxygenated blood to supply endangered cells with the resources they need to prevent irreversible damage. In cases of a stroke, a needle could be introduced into the spinal canal and viruses transporting vital proteins, nutrients, or ATP molecules could be injected through the needle. The virus virions could traverse the spinal fluid as an alternate means of reaching brain cells rather than by blood vessels and deliver to specific brain cells their payload of vital proteins, nutrients, or ATP molecules. Such an action could provide minutes to hours of life saving support to brain tissues that are in danger of irreversible damage. In the case of a serious life-threatening heart attack, a needle could be passed through the chest wall to the heart and viruses transporting vital proteins, nutrients, or ATP molecules could be injected through needle directly into heart muscle tissue. The virus virions could traverse heart tissues as an alternate means of delivering to heart cells their payload of vital proteins, nutrients, or ATP molecules. Such an action could provide minutes to hours of life saving support to heart tissues that are in danger of irreversible damage. In the case of diabetic crisis, many tissues throughout the body suffer from a dysfunction of glucose metabolism. Virus virions could be introduced into the blood stream fashioned with probes to seek out and engage the tissues of vital organs such as the brain, heart and kidneys to deliver vital proteins, nutrients, or ATP molecules to these tissues until the diabetic crisis has been resolved. In cases of severe oxygen depravation, the method of utilizing modified viruses to transport the enzyme lactate dehydrogenase to cells in crisis could result in an increase in anaerobic respiration in such cells thus providing cells with some energy production despite a lack of oxygen, to increase their survivability.

### BRIEF SUMMARY OF THE INVENTION

A medical treatment method which involves modified virus virions to be used as a transport medium to carry a payload of medically therapeutic protein molecules, nutrient molecules or energy molecules to specific cells in the body. The modified virus virions makes contact with target cells by means of the modified virus virions' exterior probes. Once the modified virus virions' exterior probes engage the cell-surface receptors of target cells the modified virus virions inserts into the target cells their payload of medically therapeutic protein molecules, nutrient molecules or energy molecules. Medical conditions such as heart attack, stroke and diabetic crisis are a result of inadequate aerobic respiration due to a lack of oxygen or proper glucose metabolism. The method by utilizing modified virus virions to transport medically therapeutic protein molecules, nutrient molecules or energy molecules to specific cells in the body may support cells in the time of crisis and improve the survivability and functionality of such cells.

### DETAILED DESCRIPTION

The medical condition where a heart attack or a stroke occurs generally is associated with or the result of a lack of sufficient supply of oxygenated blood to the heart in the case of a heart attack or to the brain in case of a stroke. Diabetic crisis occurs due to cells not being able to sufficiently utilize circulating glucose. All of these conditions may lead to irreversible damage to cells in the body.

The Hepatitis C virus (HCV) is comprised of an outer lipoprotein envelope and an internal nucleocapsid. The virus's genetic payload is carried within the nucleocapsid. The HCV's naturally occurring genetic payload consists of a single molecule of linear positive sense, single stranded RNA approximately 9600 nucleotides in length, which includes: the structural proteins of C, E1, E2, the nonstructural proteins NS1, NS2, NS3, NS4A, NS4B, NS5A, NS5B, p7 and ARFP/F protein. Present on the surface of the outer envelope of the Hepatitis C virus are probes that detect receptors present on the surface of liver cells. The glycoproteins E1 and E2 have been identified to be affixed to the surface of HCV. Portions of the Hepatitis C virus genome, when separated into individual pieces, behave like messenger RNA. Naturally occurring HCV is constructed with surface probes fashioned to recognize a receptor on the surface of a liver cell. Once the naturally occurring HCV's surface probe E2 engages a liver cell's CD81 receptor, and cofactors on the surface of HCV's exterior envelope engage the low density lipoprotein receptor (LDLR) or the scavenger receptor class B type I (SR-BI) on the liver cell, HCV then has the opportunity to insert its RNA genetic payload into the engaged target liver cell. The Human Immunodeficiency Virus seeks out and engages its host, the T-Helper cell, by utilizing its glycoprotein 120 probe and glycoprotein 41 probe to intercept the CD4 cell-surface receptor and either the CCR5 or CXCR4 cell-surface receptor on the T-Helper cell. Modifying Hepatitis C virus virions to carry therapeutic protein molecules, nutrient molecules, or energy molecules as its payload rather than its own innate genome and modifying the Hepatitis C virus's external probes to target specific cells in the body provides a medically therapeutic method to treat individuals that may potentially suffer from a heart attack, stroke or diabetic crisis. Creating virus-like structures to carry therapeutic protein molecules, nutrient molecules, or energy molecules and constructing the virus-like structures to have affixed to their surface external probes to target specific cells in the body also provides a medically therapeutic method to treat individuals that may potentially suffer from a heart attack, stroke or diabetic crisis.

Replicating viruses and constructing viruses to carry DNA payloads is a form of manufacturing technology that has already been well established and is in use facilitating gene therapy. Replicating viruses and constructing these viruses to carry messenger ribonucleic acid as the genetic payload would incorporate similar techniques as already proven useful in current gene therapy technologies.

To carry out the process to manufacture a modified medically therapeutic Hepatitis C virus, messenger RNA that would code for the general physical outer structures of the Hepatitis C virus would be inserted into a host. The host may include devices such as a host cell or a hybrid host cell. The host may utilize DNA or RNA or a combination of genetic instructions in order to accomplish the construction of medically therapeutic modified virus virions. The DNA or messenger RNA molecules to create the medically therapeutic hepatitis virus would direct the cells to generate copies of the medically therapeutic virus carrying a medically therapeutic mRNA payload. In some cases DNA or messenger RNA would be inserted into the host that would be coded to cause the production of surface probes that would be affixed to the surface of the virus virion that would target the surface receptors on specific cells in the body other than the liver cells the Hepatitis C virus naturally targets. DNA or messenger RNA would direct the host to generate copies of the proteins that would provide a therapeutic action, these medically therapeutic proteins would take the place of the Hepatitis C virus's innate genome as its payload. DNA or messenger RNA would direct the host to generate a quantity of the nutrient molecules or energy molecules that would provide a therapeutic action, these medically therapeutic nutrient molecules or energy molecules would take the place of the Hepatitis C virus's innate genome as its payload. Alternatively, artificially, a quantity of protein molecules, nutrient molecules or energy molecules could be inserted into the host so that these medically therapeutic protein molecules, nutrient molecules or energy molecules would take the place of the Hepatitis C virus's innate genome as its payload. The medical treatment form of the Hepatitis C virus carrying the medically therapeutic messenger RNA would be produced, assembled and released from a host. Virus-like structures would be generated in similar fashion using a host such as host-cells or hybrid host cells. The copies of the medically therapeutic hepatitis virus or virus-like structures, upon exiting the host, would be collected, stored and utilized as a medical treatment as necessary.

To treat tissues suffering from a lack of oxygen in times of a heart attack or a stroke, or times where glucose metabolism is dysfunctional such as in a diabetic crisis modified Hepatitis C virus or virus-like structures could be used to transport medically therapeutic protein molecules, nutrient molecules or energy molecules to specific cells in the body to improve glucose metabolism including glycolysis, tricarboxylic acid cycle and oxidative phosphorylation or cause the cell to engage in anaerobic respiration.

The modified Hepatitis C virus or virus-like structures would be incapable of replication on its own due to the fact that the messenger RNA that a naturally occurring Hepatitis C virus would normally carry would not be present in the modified form of the Hepatitis C virus or virus-like structure.

The modified Hepatitis C virus virions or virus-like structures could be fashioned to carry nutrient molecules such as oxygen molecules, glucose molecules, fatty acid molecules, vitamin molecules or mineral molecules. The modified Hepatitis C virus virions or virus-like structures could be fashioned to carry energy molecules such as adenosine triphosphate molecules, adenosine diphosphate molecules, nicotinamide adenine dinucleotide molecules, reduced form of nicotinamide adenine dinucleotide molecules, flavin adenine dinucleotide molecules, or reduced form of flavin adenine dinucleotide molecules. The modified Hepatitis C virus virions or virus-like structures could be fashioned to carry enzymes such as those described in the following paragraphs.

Hexokinase (EC 2.7.1.1) also referred to as hexokinase type IV glucokinase or simply glucokinase. Hexokinase converts glucose to glucose-6-phosphate in the process of glycolysis.

Glucose-6-phosphate isomerase (EC 5.3.1.9) also known as glucose-6-phosphate isomerase. Glucose-6-phosphate isomerase is an enzyme that converts glucose-6-phosphate to fructose-6-phosphate in the process of glycolysis.

Phosphofructokinase (EC 2.7.1.11) also known as 6-phosphofructokinase. Phosphofructokinase is an enzyme that converts fructose-6-phosphate to Fructose 1,6-diphosphate in the process of glycolysis.

Fructose bisphosphate aldolase (EC 4.1.2.13), also known as aldolase. Fructose bisphosphate aldolase is an enzyme that converts fructose 1,6-diphosphate to two different entities including dihydroxyacetone 3-phosphate and glyceraldehydes 3-phosphate in the process of glycolysis.

Triose-phosphate dehydrogenase (EC 5.3.1.1). Triose-phosphate dehydrogenase is an enzyme that converts glyceraldehydes 3-phosphat to 1,3-diphosphoglycerate in the process of glycolysis.

Phosphoglycerate kinase (EC 2.7.2.3). Phosphoglycerate kinase is an enzyme that converts 1,3-diphosphoglycerate to 3-phosphoglycerate in the process of glycolysis.

Phosphoglycerate mutase (EC 5.4.2.1). Phosphoglycerate mutase is an enzyme that converts 3-phosphoglycerate to 2-phosphoglycerate in the process of glycolysis.

Enolase (EC 4.2.1.11). Enolase is an enzyme that converts 2-phosphoglycerate to phosphoenolpyruvate in the process of glycolysis.

Pyruvate kinase (EC 2.7.2.3). Pyruvate kinase is an enzyme that converts phosphoenolpyruvate to pyruvate in the process of glycolysis.

Pyruvate dehydrogenase. Pyruvate dehydrogenase is comprised of three units. The three units include E1 (EC 1.2.4.1), (EC 1.2.1.51), E2 dihydrolipoamide S-acetyltransferase (EC 2.3.1.12), and E3 dihydrolipoamide dehydrogenase (EC 1.8.1.4). Pyruvate dehydrogenase molecular complex catalyzes the conversion of pyruvate to acetyl-CoA.

Citrate synthetase (EC 4.1.3.7). Citrate synthetase is an enzyme that converts acetyl-CoA combines with oxaloacetate to produce citrate in the tricarboxylic acid cycle.

Aconitase (EC 4.2.1.3). Aconitase is an enzyme that converts citrate to cis-aconitate in the tricarboxylic acid cycle. Aconitase is an enzyme that converts cis-aconitate to iso-citrate in the tricarboxylic acid cycle.

Isocitrate dehydrogenase (EC 1.1.1.41). Isocitrate dehydrogenase is an enzyme that converts isocitrate to alpha-ketoglutaric acid in the tricarboxylic acid cycle.

2-oxoglutarate dehydrogenase. 2-oxoglutarate dehydrogenase is protein complex comprised of three units. The three units include E1 (EC 1.2.4.2), E2 (EC 2.3.1.61), and E3 (EC 1.8.1.4). 2-oxoglutarate dehydrogenase is an enzyme complex that converts alpha-ketoglutaric to succinyl-CoA in the tricarboxylic acid cycle.

Succinyl-CoA synthetase (EC 6.2.1.5). Succinyl-CoA synthetase is an enzyme that converts succinyl CoA to succinate in the tricarboxylic acid cycle.

Succinate dehydrogenase (EC 1.3.5.1). Succinate dehydrogenase is an enzyme that converts succinate to fumarate in the tricarboxylic acid cycle.

Fumarate hydratase (EC 4.2.1.2). Fumarate hydratase is an enzyme that converts fumarate to malate in the tricarboxylic acid cycle.

Malate dehydrogenase (EC 1.1.1.37). Malate dehydrogenase is an enzyme that converts malate to oxaloacetate in the tricarboxylic acid cycle.

NADH dehydrogenase (EC 1.6.5.3) molecule, also referred to as NADH-coenzyme Q oxidoreductase or complex I, is utilized in oxidative phosphorylation.

Succinate dehydrogenase (EC 1.3.5.1) molecule, also referred to as succinate oxidoreductase or complex II, is utilized in oxidative phosphorylation.

Cytochrome-c reductase (EC 1.10.2.2) molecule, also referred to as complex III, is utilized in oxidative phosphorylation.

Cytochrome-c oxidase (EC 1.9.3.1) molecule, also referred to as complex IV, is utilized in oxidative phosphorylation.

ATP synthase (EC 3.6.1.34) molecule is utilized in oxidative phosphorylation.

Lactate dehydrogenase (EC 1.1.1.27) molecule is utilized to convert pyruvate to lactic acid in anaerobic respiration.

In review, the medical treatment method described in this text includes taking a naturally occurring virus and altering its payload so that it transports medically therapeutic protein molecules, nutrient molecules, or energy molecules to cells it was naturally designed to infect, but instead of delivering its own genetic payload, it delivers the medically therapeutic protein molecules, nutrient molecules, or energy molecules it is carrying, and the medical treatment method described in this text includes taking a naturally occurring virus and altering its payload so that it carries medically therapeutic protein molecules, nutrient molecules, or energy molecules and alter the virus's glycoprotein probes so that it is capable of infecting specifically targeted cells, but instead of delivering its own genetic payload, it delivers to specific cells the medically therapeutic protein molecules, nutrient molecules, or energy molecules it is carrying, and the medical treatment method described in this text includes taking a virus-like structure, which carries medically therapeutic protein molecules, nutrient molecules, or energy molecules, affixed to the surface glycoprotein probes so that it is capable of delivering medically therapeutic protein molecules, nutrient molecules, or energy molecules it is carrying to specific target cells to produce a beneficial medically therapeutic outcome.

As mentioned above, the medical treatment method to improve glucose metabolism during the threat of a heart attack, stroke, or diabetic crisis, involves a quantity of modified virus virions, such as modified Hepatitis C virus virions or virus-like structures that would be introduced into a patient's blood stream, or into spinal fluid, or directly into the endangered tissues so that the modified virus could deliver the medically therapeutic mRNA payload that it carries to targeted cells in the body. Once the modified virus virions or virus-like structures insert their medically therapeutic payload consisting of medically therapeutic protein molecules, nutrient molecules, energy molecules into the cells the modified virus virions or virus-like structures have been targeted for, the cells' biologic function of metabolizing glucose by way of glycolysis, tricarboxylic acid cycle, oxidative phosphorylation or anaerobic respiration will be enhanced. Improvement in the metabolism of glucose inside cells and the resultant increase in production of energy molecules will reduce the risk of irreversible damage to cells during conditions when oxygen depravation threatens cells of the body.

### DRAWING

None.

What is claimed:

1. A medical treatment method for inserting a quantity of protein molecules into cells of the body comprising:
(a) a quantity of modified virus virions generated for the purpose of carrying a quantity of said protein molecules,
(b) the quantity of said virus virions having glycoprotein probes affixed to the surface, said glycoprotein probes constructed in a manner to target specific cells in said body,
(c) said glycoprotein probes capable of engaging specific cell-surface receptors on said cells,
(d) once said glycoprotein probes have successfully engaged cell-surface receptors on said cells, the quantity of said modified virus virions deliver into said cells a quantity of said protein molecules said modified virus virions are carrying,
whereby said cells will be made capable of engaging in biologic processes which said protein molecules are necessary as a catalyst, for purposes of accomplishing a medical treatment,
whereby said cells' biochemistry capacity will be improved regarding engaging in biologic processes which said protein molecules are necessary as a catalyst, for purposes of accomplishing a medical treatment.

2. The medical treatment method in claim 1 wherein said modified virus virions selected from the group consisting of naturally occurring virus virions whose payload has been altered to carry a quantity of protein molecules, naturally occurring virus virions whose payload has been altered to carry a quantity of protein molecules said virus virions capable of delivering said quantity of protein molecules which the surface glycoprotein probes have been altered in a manner the glycoprotein probes are fashioned to engage specific cells, and virus-like structures constructed to resemble a naturally occurring virus virions said virus-like structures capable of carrying a quantity of protein molecules said virus-like structures capable of delivering said quantity of said protein molecules said virus-like structures constructed with glycoprotein probes fashioned to engage specific cells.

3. The medical treatment method in claim 1 wherein said wherein said modified virus virions selected from the group consisting of Hepatitis A virus virions, Hepatitis B virus virions, Hepatitis C virus virions, Hepatitis D virus virions, Hepatitis F virus virions, Hepatitis E virus virions, Hepatitis G virus virions, and Hepatitis H virus virions.

4. The medical treatment method in claim 1 wherein said protein molecules selected from the group consisting of those enzymes that participate in the biologic process of glycolysis, those enzymes that participate in the biologic process of the tricarboxylic acid cycle, those enzymes that participate in the biologic process of oxidative phosporylation, and those enzymes that participate in the biologic process of anaerobic respiration.

5. The medical treatment method in claim 1 wherein said protein molecules selected from the group consisting of
hexokinase Enzyme Commission of the International Union of Biochemistry (EC) 2.7.1.1 enzyme molecules,
glucose-6-phosphophate isomerase EC 5.3.1.9 enzyme molecules,
phosphofructo kinase EC 2.7.1.11 enzyme molecules,
fructose biphosphate aldolase EC 4.1.2.13 enzyme molecules,
triose-phosphate isomerase EC 5.3.1.1 enzyme molecules,
glyceraldehyde-3-phosphate dehydrogenase EC 1.2.1.12 enzyme molecules,
phosphoglycerate kinase EC 2.7.2.3 enzyme molecules,
phosphoglycerate mutase EC 5.4.2.1 enzyme molecules,
enolase EC 4.2.1.11 enzyme molecules,
pyruvate kinase EC 2.7.1.40 enzyme molecules,
pyruvate dehydrogenase (acetyl-transferring) EC 1.2.4.1 enzyme molecules,
pyruvate dehydrogenase (NADP+) EC 1.2.1.51 enzyme molecules,
dihydrolipoamide S-acetyltransferase EC 2.3.1.12 enzyme molecules,
dihydrolipoamide dehydrogenase EC 1.8.1.4 enzyme molecules,
citrate synthetase EC 4.1.3.7 enzyme molecules,
aconitase EC 4.2.1.3 enzyme molecules,
isocitrate dehydrogenase EC 1.1.1.41 enzyme molecules,
2-oxoglutarate dehydrogenase E1 EC 1.2.4.2 enzyme molecules,
2-oxoglutarate dehydrogenase E2 EC 2.3.1.61 enzyme molecules,
2-oxoglutarate dehydrogenase E3 EC 1.8.1.4 enzyme molecules,
succinyl-CoA synthetase EC 6.2.1.5 enzyme molecules,
succinate dehydrogenase EC 1.3.5.1 enzyme molecules,
fumarate hydratase EC 4.2.1.2 enzyme molecules,
malate dehydrogenase EC 1.1.1.37 enzyme molecules,
nicotinamide adenine dinucleotide (NADH) dehydrogenase EC 1.6.5.3 enzyme molecules,
succinate dehydrogenase EC 1.3.5.1 enzyme molecules,
cytochrome-c reductase EC 1.10.2.2 enzyme molecules,
cytochrome-c oxidase EC 1.9.3.1 enzyme molecules,
ATP synthase EC 3.6.1.34 enzyme molecules, and
lactate dehydrogenase EC 1.1.1.27 enzyme molecules.

6. The medical treatment method in claim 5 wherein said enzyme molecules are comprised of a quantity of protein subunits to create a protein complex.

7. The medical treatment method in claim 1 wherein said body is comprised of the physical features of the human body.

8. A medical treatment method for inserting a quantity of nutrient molecules into cells of the body comprising:
(a) a quantity of modified virus virions generated for the purpose of carrying a quantity of said nutrient molecules,
(b) the quantity of said modified virus virions having glycoprotein probes affixed to the surface, said glycoprotein probes constructed in a manner to target specific cells in said body,
(c) said glycoprotein probes capable of engaging specific cell-surface receptors on said cells,
(d) once said glycoprotein probes have successfully engaged cell-surface receptors on said cells, the quantity of said modified virus virions deliver into said cells a quantity of said nutrient molecules said modified virus virions are carrying,
whereby said cells will be made capable of engaging in biologic processes which said nutrient molecules are necessary, for purposes of accomplishing a medical treatment,
whereby said cells' biochemistry capacity will be improved regarding engaging in biologic processes which said nutrient molecules are necessary, for purposes of accomplishing a medical treatment.

9. The medical treatment method in claim 8 wherein said nutrient molecules selected from a group consisting of oxygen molecules, glucose molecules, fatty acid molecules, vitamin molecules and mineral molecules.

10. The medical treatment method in claim 8 wherein said modified virus virions selected from the group consisting of naturally occurring virus virions whose payload has been altered to carry a quantity of nutrient molecules, naturally occurring virus virions whose payload has been altered to carry a quantity of nutrient molecules said modified virus virions capable of delivering said quantity of nutrient molecules which the surface glycoprotein probes have been altered in a manner the glycoprotein probes are fashioned to engage specific cells, and virus-like structures constructed to resemble naturally occurring virus virions said virus-like structures capable of carrying a quantity of nutrient molecules said virus-like structures capable of delivering said quantity of nutrient molecules said virus-like structures constructed with glycoprotein probes fashioned to engage specific cells.

11. The medical treatment method in claim 8 wherein said modified virus virions selected from the group consisting of Hepatitis A virus virions, Hepatitis B virus virions, Hepatitis C virus virions, Hepatitis D virus virions, Hepatitis F virus virions, Hepatitis E virus virions, Hepatitis G virus virions, and Hepatitis H virus virions.

12. The medical treatment method in claim 8 wherein said body is comprised of the physical features of the human body.

13. A medical treatment method for inserting a quantity of energy molecules into cells of the body comprising:
(a) a quantity of modified virus virions generated for the purpose of carrying a quantity of said energy molecules,
(b) the quantity of said modified virus virions having glycoprotein probes affixed to the surface, said glycoprotein probes constructed in a manner to target specific cells in said body,
(c) said glycoprotein probes capable of engaging specific cell-surface receptors on said cells,
(d) once said glycoprotein probes have successfully engaged cell-surface receptors on said cells, the quantity of said modified virus virions deliver into said cells a quantity of said energy molecules said modified virus virions are carrying,
whereby said cells will be made capable of engaging in biologic processes which said energy molecules are necessary as an energy source, for purposes of accomplishing a medical treatment,
whereby said cells' biochemical capacity will be improved regarding engaging in biologic processes which said energy molecules are necessary as an energy source for purposes of accomplishing a medical treatment.

14. The medical treatment method in claim 13 wherein said energy molecules selected from the group consisting of adenosine triphosphate molecules, adenosine diphosphate molecules, nicotinamide adenine dinucleotide molecules, reduced form of nicotinamide adenine dinucleotide molecules, flavin adenine dinucleotide molecules, and reduced form of flavin adenine dinucleotide molecules.

15. The medical treatment method in claim 13 wherein said modified virus virions selected from the group consisting of naturally occurring virus virions whose payload has been altered to carry a quantity of energy molecules, naturally occurring virus virions whose payload has been altered to carry a quantity of energy molecules said modified virus virions capable of delivering said quantity of energy molecules which the surface glycoprotein probes have been altered in a manner the glycoprotein probes are fashioned to engage specific cells, and virus-like structures constructed to resemble naturally occurring virus virions said virus-like structures capable of carrying a quantity of energy molecules said virus-like structures capable of delivering said quantity of energy molecules said virus-like structures constructed with glycoprotein probes fashioned to engage specific cells.

16. The medical treatment method in claim 13 wherein said modified virus virions selected from the group consisting of Hepatitis A virus virions, Hepatitis B virus virions, Hepatitis C virus virions, Hepatitis D virus virions, Hepatitis F virus virions, Hepatitis E virus virions, Hepatitis G virus virions, and Hepatitis H virus virions.

17. The medical treatment method in claim 13 wherein said specific cells selected from the group consisting of cells comprising the muscles, cells comprising the brain, cells comprising the heart, cells comprising the pancreas, cells comprising the endocrine glands, cells comprising the dermis, cells comprising the mucosa, cells comprising the gastroenteric tract, cells comprising the renal system, cells comprising the skeletal structure, cells comprising the pulmonary system, cells comprising the nerve system, cells comprising the immune system, cells comprising the sex organs, cells comprising the spleen, and cells comprising the liver.

18. The medical treatment method in claim 13 wherein said body is comprised of the physical features of the human body.
